(19) United States
(12) Patent Application Publication
(10) Pub. No.: US 2003/0040027 A1
(43) Pub. Date: Feb. 27, 2003

(54) Method for determining protein component in a biological sample

(76) Inventors: Gerd Ritter, New York, NY (US); Leonard S. Cohen, New York, NY (US); Lloyd Old, New York, NY (US); Sydney Welt, New York, NY (US)

Correspondence Address: FULBRIGHT & JAWORSKI, LLP, 666 FIFTH AVE, NEW YORK, NY, 10103-3198, US

(21) Appl. No.: 10/222,319

(22) Filed: Aug. 16, 2002

Related U.S. Application Data
(60) Provisional application No. 60/312,976, filed on Aug. 16, 2001.

(51) Int. Cl. G01N 33/567; G01N 33/574; G01N 33/53; G01N 33/542

(52) U.S. Cl. 435/7.21; 435/7.9; 435/7.23

(57) ABSTRACT

Plasmon resonance is used to determine presence of immunoactive molecules, such as antibodies, against therapeutic agents administered to subjects. The determination can be used as an indicia of therapeutic efficacy lack thereof, or problems resulting from the agent.

### RELATED APPLICATION

This applications claims priority of application Serial No. 60/312,976, filed Aug. 16, 2001, incorporated by reference.

### FIELD OF THE INVENTION

This invention relates to methods for determining proteins in a biological sample, such as blood or plasma, such as antibodies which are produced as part of an adverse reaction to antibodies when the antibodies in question are used therapeutically. More specifically, it relates to detection of human anti human antibodies, (HAHA) or human, anti-chimerized antibodies (HACA) in a biological sample, such as blood plasma, serum, urine, cerbrospinal fluid, sweat, tissue exudates, tissue homogenates, in subjects who received, e.g., humanized antibodies, fully human antibodies, chimerized antibodies, or any kind of fragments, or multimers of fragments thereof, such as, but not being limited to, Fab, F(ab′)₂, Fv, scFv, disulfide linked Fv, dia-, tria-, tetra- and other multimeric antibodies, and so forth, as part of a clinical protocol. Even more specifically, it relates to determining a HAHA response against humanized A33 specific antibodies, using a BIACORE® (surface plasmon resonance) system. The method permits the artisan to determine when severe adverse events, such as infusion related events; are imminent, thereby allowing for better monitoring of therapeutic methods. In a further aspect of the invention, it relates to a method for determining progress of a therapeutic regime by determining levels of a protein, such as an antibody, at different points during the regime and comparing them.

The use of proteins as therapeutic agents has a long history which will be familiar to the artisan, and need not be recited herein. Examples of proteins used therapeutically include insulin, GM-CSF, erythropoietin, tissue plasminogen activator, nerve growth factor and many others. One family of proteins that has been used therapeutically are antibodies, including monoclonal antibodies, humanized antibodies, as well as antibody fragments and antibody oligomers. While the discussion which follows uses adverse reaction to antibodies, monoclonal and/or humanized antibodies in particular as exemplary of the invention, it is to be understood that adverse reactions can and do occur against proteins used therapeutically. Any discussion of the “HAHA” or “HAMA” response presented herein is to be interpreted as referring analogously to any antibody response to any agent, with respect to the determination thereof.

### BACKGROUND AND PRIOR ART

Monoclonal antibodies, or “mAbs” hereafter, have become recognized as effective agents in the treatment of pathological conditions, including cancer.

Originally, the mAbs which were used for treatment were murine, or mouse based monoclonal antibodies. While these showed efficacy, a problem developed, known as the “human anti mouse antibody” or “HAMA” response. In brief, the HAMA response is the human immune system's response to murine mAbs, which are recognized as foreign molecules. The response, which develops over time, occurs after only a few injections of the potentially therapeutically useful mAb.

One attempt to alleviate or to eliminate the HAMA response involves the development and use of humanized antibodies. Such humanized antibodies consist of a portion of a human antibody, such as the framework region, onto which a non-human portion, such as a murine CDR region, is grafted. The theory underlying administration of such humanized antibodies is that, as they are very close to naturally occurring human antibodies, they will evade surveillance by the subject's immune system.

In practice, the use of humanized antibodies has permitted longer therapeutic time frames, but a human anti human antibody, or “HAHA” response, is still observed.

The history of antibody therapy against the antigen referred to as “A33” is illustrative. The A33 antigen is a cell surface differentiation antigen of normal human gastrointestinal epithelium. The antigen is expressed in 95% of primary and metastatic colon cancer cells, but is essentially absent in most other normal tissues and tumor types. See, e.g., Welt, et al., J.Clin Oncol 8:1894-1906 (1990); Garin-Chesa, et al., Int. J. Oncol 9:465-471 (1996). The antigen has been characterized as a 43kd, transmembrane glycoprotein, and a member of the Ig superfamily. See Heath, et al., Proc. Natl. Acad Sci USA 94:469-474 (1997); Catimel, et al., J. Biol Chem 271:25664-25670 (1996); Chretien, et al., Eur. J. Immunol 28:4094-4104 (1998). There is some distant relationship to a lymphocyte differentiation antigen of Xenopus (Chretien, et al., Eur. J. Immunol 26:780-791 (1996), as well as a cocksackie virus, and an adenovirus receptor. See Bergelson, et al., Science 272:1320-1323 (1997). Further analysis showed that the antigen consists of two extra-cellular Ig domains, a single transmembrane domain, and a short, intracellular tail which contains four (4) acylation sites proximal to the transmembrane domain. See, e.g., Heath, et al., supra; Ritter, et al., Biochem. Biophys. Res. Commun 236:682-686 (1997). The molecule is not secreted, nor is it shed, into the blood. Some colon cancer cell lines express large amounts of the antigen. Indeed, up to 800,000 molecules of antibodies specific for the antigen have been found to bind to some cells. See, e.g., Daghigian, et al., J. Nucl. Med 37:1052-1057 (1996).

The murine mAb which is also referred to as “A33” binds to this antigen, and localizes, with high specificity to colon cancer in patients. The mAb is retained for up to six (6) weeks in cancer, but is cleared in 5-6 days from normal colon tissue. When labeled with either ¹²⁵I or ¹³¹I, the murine mAb exhibits anti-cancer activity. See Welt, et al., J. Clin. Oncol 8:1894-1906 (1990); Welt, et al., J. Clin. Oncol 12:1561-1571 (1994); Welt, et al., J. Clin. Oncol 14:1787-1797 (1996). Strong HAMA responses have, however, curtailed it use.

King, et al., Br. J. Cancer 72:1364-1372 (1995) describe preparation of a humanized A33 antibody, where the murine CDR of A33 was grafted onto a human IgG1 framework. The humanized antibody retained full binding specificity and affinity. Clinical trials, both phase I and phase II, have been carried out in patients with colon cancer, using the humanized antibody.

It has now been found, however, that one can detect a HAHA response against these humanized antibodies in patients receiving the drug, permitting the artisan to monitor therapeutic regimes more closely and to develop appropriate therapeutic courses. These, as well as other feature of the invention, will be seen in the disclosure which follows.

### DETAILED DESCRIPTION OF PREFERRED EMBODIMENTS

### EXAMPLE 1

The humanized antibody “HuAb A33” is a fully humanized, IgG1 monoclonal antibody. Its production is described by, e.g., King, et al., Br J. Cancer 72:1364:1372 (1995), the disclosure of which is incorporated by reference. In brief, the humanized antibody was prepared by CDR grafting onto known murine monoclonal antibody mAb A33. The antibody was expressed in NSO cells, and was recovered from NSO culture supernatant using, in sequential order, Q sepharose anion exchange, protein A affinity, and S Sepharose action exchange chromatography Virus inactivation was carried out, via standard methods, and the purified antibody was stored at −80° C. Purified antibody was tested, when thawed, and it retained the original specificity and avidity.

Additional antibodies were also used in the experiments which follow. Humanized 3S193 antibody, which is an IgG1 antibody that binds to Lewis Y antigen, was prepared in accordance with Kitamura, et al., Proc. Natl. Acad Sci USA 91:12957-12961 (1994), and Scott, et al., Canc. Res. 60:3254-3261 (2000). Murine antibodies “A33” and 100.310, both of which are IgG2a, were prepared in accordance with Welt, et al., J. Clin. Oncol 8:1894-1906 (1990). Pegylated humanized mAb A33 was prepared in accordance with Deckert, et al, Int. J. Cancer 87:382-390 (2000), while SK10B, a humanized IgG1 with the same allotype as humanized mAb A33, and humanized antibody SK10C (IgG4), were purchased. A human-mouse chimeric, IgG1 antibody (G250), was purchased, and is described by Oosterwijk, et al, Int. J. Canc. 38:489-494 (1996).

### EXAMPLE 2

These experiments describe initial BIACORE analysis on samples taken from patients receiving humanized mAb A33, according to one of the protocols listed infra.

Forty-four patients were studied. In each case, blood samples were taken from patients immediately before administration of the antibodies. Sera was separated from the blood samples, using standard methods, and was used in the assays which are described herein. The serum samples were analyzed via surface plasmon resonance technology using a BIACORE 2000 instrument. In brief, humanized antibodies in solutions of 5 mM sodium phosphate, pH 5.5, or murine antibodies, in 6 mM sodium acetate, pH 4.6, were immobilized to CM5 biosensor chips, using N-hydroxy-succinimide (“NHS”), -ethyl-N′-(dimethyl aminoprophy) carbodiimide. About 10,000, ±2,000 Response Units were immobilized per flow cell, in order to carry out HAHA measurements.

Microchips sensor surfaces were conditioned with three cycles of 5× injections of each of 5 μl 15 mM HCl, followed by 1× injection of 10 μl 0.2M Na₂ CO₃ buffer containing 1MNaCl, pH10 prior to use.

Patient serum was diluted with HBS-buffer (10 mM HEPES, 0.15M sodium chloride, 3.4 mM EDTA, and 0.05% surfactant) containing 1 mg/ml carboxymethyldextran, pH 7.4, and was then passed over the chips. Following this, the chips were washed with HBS containing 0.5M NaCl, pH 7.4. Alterations in the refractory index were recorded as relative Response Units, in accordance with manufacturer guidelines. The antibody binding was given in RU over baseline, recorded 70 seconds after injection of the wash buffer. Baseline values were recorded 10 seconds prior to sample injection, and were set at 0 RUs. The microchip sensor surfaces were regenerated with 10 μl of 15 mM HCl prior to each new injection cycle. A patient serum sample was considered HAHA if the RU value at a serum dilution of 1:100 exceeded a cut-off value, which was defined as the mean, inter-patient RU value+3XSD (standard deviation) of pre-treatment sera at a dilution of 1:100.

Prior to treatment, none of the 44 patients exhibited a measurable HAHA response. The median response level was 14 RU. Following treatment, however, 20/28 of the patients treated with humanized mAb A33 alone exhibited a HAHA response. This was divided into 8/11 patients in the phase I trial, and 12/17 in the phase II trial (68%). Of the 13 patients receiving the humanized antibody and chemotherapy, 6 exhibited a response (46%). All three patients receiving ¹³¹I conjugated humanized antibody exhibited a HAHA response. The values ranged from 30 up to 1500 RUs, following treatment. The HAHA response was detectable as early as one week after the first infusion. There did not appear to be any correlation between onset or strength of the response and dosage. Patients who received the antibody in connection with chemotherapy appeared to develop the response at a lower frequency (46%) and had lower peak HAHA levels (median peak: 48 RU, ranging from 32-831 RU), as compared to those who received the antibody alone (frequency: 68%; median peak: 75 RU; range: 30-1503 RU). Approximately 37% (15/41) of patients did not develop HAHA, or had only a marginal, temporary increase in levels.

Analysis of the data showed two distinct patterns of HAHA response. The first pattern is characterized by early onset of HAHA, with levels peaking after about two weeks of treatment, followed by decline of measurable levels even though treatment was continued. Approximately, 49% (20/41) of patients showed this pattern. Peak HAHA levels, after two rounds of administration, ranged from high (increase >100 RU, 4/41 patients), medium high (increase: 41-100 RU; 5 patients), and low (increase: 20-40 RU, 10 patients). All three patients treated with ¹³¹I labeled humanized A33 mAb developed this pattern of response. Two of the patients had high, and one had medium high levels.

The second response pattern showed delayed onset of HAHA, with an increase following subsequent treatment About 17% (7/41) patients showed this pattern. Using the RU ranges, supra, three (3) patients showed high levels, three (3) showed medium high levels, and one showed a low level increase.

As a result of this pattern, the treatment was discontinued in these patients. This was also he case for a patient receiving the antibody plus chemotherapy, who developed a type I response, and subsequently developed a type II response.

TABLE 1
Summary of treatment schedules for huAb A33
in patients with colon cancer

| | HuAb A33 DOSE | |
|---|---|---|
| PATIENT | mg Ig/m² | TREATMENT SCHEDULE |
| COL-1-5 | 10 | cycles (4 weeks, 1 x antibody |
| | | infusion per week, followed by |
| | | 1 week off) |
| COL-6-8 | 25 | cycles (4 weeks, 1 x antibody |
| | | infusion per week, followed by |
| | | 1 week off) |
| COL-9-11 | 50 | cycles (4 weeks, 1 x antibody |
| | | infusion per week, followed by |
| | | 1 week off) |
| COL-12-28 | 10 | 10 weeks, 1 x antibody infusion |
| | | per week |
| COL-29, 37, 39 | 5 | 14 weeks, 1 x antibody infusion |
| | | per week, BOF-Strep, week 5- |
| | | 14, once per week |
| COL-30, 33, 35, 38 | 10 | 14 weeks, 1 x antibody infusion |
| | | per week, BOF-Strep, week 5- |
| | | 14, once per week |
| COL-31, 32, 34, 36 | 25 | 14 weeks, 1 x antibody infusion |
| | | per week, BOF-Strep, week 5- |
| | | 14, once per week |
| COL-40, 41 | 40 | 14 weeks, 1 x antibody infusion |
| | | per week, BOF-Strep, week 5- |
| | | 14, once per week |
| COL-42-44 | 10 | [131]-huAb A33, 1x dose per |
| | | week |

### EXAMPLE 3

The assay described in example 2 measures direct interaction of serum components with immobilized antigen, without using secondary reagents. The experiments described herein were carried out to determine if the reactivity was due to immunoglobulin binding to the humanized antibody.

Serum samples were diluted, 1:100. Following the dilution, 500 μl samples were combined with approximately 100 μl of protein G, attached to Sepharose beads. The mixture was incubated, overnight at 4° C., on a rotating platform. Beads were removed, via centrifugation, and the serum samples, now depleted, were used in the BIACORE assays described supra. No activity was found.

Protein G precipitation removes IgG, so the results suggested that the reactivity was, in fact, caused by IgG molecules. In follow up experiments, serum samples were treated with caprylic acid, in accordance with Steinbuch, et al, Arch. Biochem. Biophys. 134:279-284 (1969), incorporated by reference. When caprylic acid is used, IgG remains in supernatants following precipitation. When the precipitates and supernatants of these experiments were tested, activity was, in fact, found in the supernatant.

There was some residual activity in the sera of two patients after the IgG was removed. The cause of the activity was found to be IgM binding, via a sandwich immunoassay for IgM carried out using BIACORE. The reactivity observed was relatively weak (an increase of <40 RU), transient (detectable in sera following protein G precipitation after 2 weeks, but not 9), and it occurred together with IgG in the early weeks of treatment. When tested in sera that had not been IgG depleted, the IgM reactivity could not be detected. This was also the case in an ELISA.

Additional experiments were carried out using anti-human IgA and IgE, and neither reacted with the antibodies bound to the humanized A33 in the BIACORE assay.

In a final set of experiments, assays were carried out in order to type the IgG molecules which bound to the humanized mAb A33. There was no binding with anti IgG2, IgG3 or IgG4. There was binding with anti-IgG1.

The experiments thus show that the increase in reactivity results from anti-human mAb A33 antibodies which are of type IgG1. There is additional, transient and weakly reactive IgM induced on occasion.

### EXAMPLE 4

This example sets forth experiments designed to carry out specificity analyses of the HAHA response.

Sera that reacted with humanized mAb A33 were diluted 1:100, and were then tested, in a BIACORE assay, for reactivity against the humanized mAb A33, unrelated antibodies or the Fab′ fragment of humanized mAb A33. The Fab′ fragments were prepared by digesting the complete antibody with papain, in accordance with manufacturer's instructions. The antibodies or Fab′ fragment were immobilized, as described in example 1, and assayed as described therein. Sera which reacted with the humanized A33 antibody did not react with human IgG1 isotype control antibodies, such as 3S193, or the murine chimeric antibody G250. Nor did they react with the unrelated humanized antibody SK10C.

In a second set of assays, patient serum was absorbed with various, humanized, mouse-human chimeric, or mouse mAbs, prior to the BIACORE assay. The antibodies were used at 50 μg/ml. Specifically, in addition to humanized mAb A33, mAbs 100.310, SK10B, and murine mAb A33 were used. The SK10B construct was used to determine potential allotypic reactivity. The humanized mAb A33 has a non-caucasian IgG1 allotype (non-2, a-1, allotype).

Only the humanized mAb A33, the Fab′ fragment of this antibody, and the murine form absorbed serum reactivity completely in all immune sera; no Ig allotype binding was seen. From these data, it can be concluded that the serum reactivity was specific for epitopes contained in humanized mAb A33, and mAb A33, and that the immunodominant epitopes are located in the variable region of the humanized antibody.

### EXAMPLE 5

In order to carry out more detailed mapping of the antigenic determinant, a series of experiments were carried out, which are summarized herein. Sera from patients who exhibited the HAHA response reacted with immobilized humanized mAb, A33 derived Fab and Fab′ fragments, but not Fe fragments. Further, these sera did not react with Fab from humanized mAbs derived from rabbits via phage display, in accordance with Rader, et al, J. Biol. Chem 275:13668-13676 (2000), incorporated by reference. Yet further, in serum absorption assays, reactivity of human patient sera against humanized mAb A33 was absorbed by the Fab′ fragment, and also a pegylated derivative which retained full A33 antigen binding activity. These results lead to the conclusion that the antigenic determinants are in the V_{L} and V_{H} regions which include the CDRs.

### EXAMPLE 6

The experiments set forth in example 6, supra led to the conclusion that the immunodominant epitopes for the humanized A33 antibodies were in the V_{L} and V_{H} region. These experiments were designed to map the epitopes to a defined sequence.

As per the citations, supra, the amino acid sequence of humanized mAb A33 is known. See e.g., King, et al, supra, incorporated by reference. A series of twenty four peptides, which corresponded to overlapping sequences in the humanized antibody V_{L} and V_{H} sequences were synthesized, in accordance with standard methods. Each of these peptides was immobilized on a PVDF membrane, and the membranes were then incubated at 4° C. overnight, in high titer sera. Any bound antibody was visualized by chemiluminescence.

Pre-immune and immune sera showed indistinguishable reactivity patterns, so mapping was not possible. Eight peptides which correspond to all humanized A33 V_{L} and V_{H} sequences containing murine residues. These were then used in BIACORE absorption assays. None blocked binding of humanized mAb A33 positive immune sera to humanized mAb A33.

Following the experiments, supra, experiments were carried out to determine if epitopes recognized by patients' sera involved glycosylated sites. To do this, humanized mAb A33 was both —N—and —O-deglycosylated, and utilized in absorption assays.

The results indicated that deglycosylated humanized mAb A33, which retained activity absorbed the reactivity of the immune sera tested, which indicates that post translational carbohydrate modifications do not contribute to the antibody's antigenicity.

In a final set of experiments, whole humanized mAb A33 was reduced with 2-mercapto-ethanol or 1,4-dithiothreitol, to produce single chains.

The reduced antibodies lost their ability to block reactivity, indicating that the main antibody response is, in fact directed against at least one, and possibly multiple, conformational antigenic determinants located within the variable heavy and light chains, requiring association of the V_{L} and V_{H} chains.

### EXAMPLE 7

As noted, supra two types of HAHA response were identified. All patients were examined for development or onset of infusion related symptoms, which include fever, chills, nausea, vomiting, rash, and myalgias. Virtually all infusion-related symptoms were observed in HAHA type II patients. This was the case even though patients with a type II HAHA response did not clearly have higher antibody titers, or RU values in the BIACORE assay, when compared to type I patients.

While the patients with type II responses did show increases in RU values over the treatment period, the symptoms did not manifest until after several weeks of progressively increasing HAHA response levels. It was thus possible to discontinue patients from the therapeutic regime, based upon the increased levels of HAHA response.

The foregoing examples set forth various aspects of the invention, which relate to methods for determining reaction to treatment with a therapeutic agent, such as a protein or other agent which stimulates an adverse response, such as an antibody, humanized antibody or fully human antibody in particular, as well as fragments thereof. As is indicated, the development of an immune response against a therapeutic agent, be it a HAMA, HAHA, HACA or other type of immune response, such as an immune response associated with, e.g., a graft-host rejection, or the administration of a human protein, for example, a form of hGH, such as met-hGH, or other human protein is undesirable; however, the usual clinical manifestations of such a response tend to occur rather late in the treatment. Hence, one aspect of the invention is a method for determining onset, existence, or continuance of a response to immunotherapy, wherein a blood or serum sample of a patient is assayed via surface plasmon resonance, to determine presence and/or quantity of reactants to the immunotherapeutic agent being administered to the patient. Preferably, the assay is carried out on serum, blood, or some other body fluid using a BIACORE device, where the patient is receiving or has received humanized antibodies, fully human or chimerized antibodies or any therapeutically useful antibody fragments.

The examples involve assays relating to cancer therapies; however, it is to be kept in mind that protein based therapies, antibodies (e.g., humanized or fully human antibodies) in particular, are available for, e.g., the treatment of conditions involving some immune componenet, such as allergies, autoimmune diseases such asrheumatoid arthritis, infections such as viral infections or prion infections, and measurement of antibody levels or other components of the immune system are features of the invention. Measurement of antibodies can also be used as a reflection of infection, such as by hepatitis C virus, or prions as well.

The invention can be used, e.g., to monitor the course of a particular mode of treatment, such as vaccination designed to confer active and/or passive immunity on the subject receiving the vaccine (“vaccine” as used herein refers to both preventive and treatment vaccines), or the course of a disease or other pathological condition. In this embodiment of the invention, one compares two or more values of a particular parameter, at different points in time, to determine if the value changes. Such a change is indicative of a change in the patient, such as a change in disease status, or a change in efficacy of treatment, etc. For example, where one is interested in generating antibodies against a pathological agent, e.g., in a vaccination protocol or some other immune stimulating protocol, efficacy can be measured by determining if antibody titer rises, as compared to a situation where one is interested in a decrease in HAHA, HAMA, or HACA response.

While the disclosure presented supra is presented in terms of carrying out a single assay at a time, it must be noted that the apparatus used in such assays are useful for measuring parameters in more than one sample at a time and the invention should not be construed as being limited to a single assay being carried out at a time.

Other aspects of the invention will be clear to the skilled artisan, and need not be developed further.

We claim:

1. In a method for determining if an immune response against a therapeutic agent has occurred by determining presence of an immune agent specific for said therapeutic agent in a sample, the improvement comprising determining presence of said immune agent via plasmon resonance.

2. The method of claim 1, wherein said therapeutic agent is a protein containing molecule.

3. The method of claim 2, wherein said protein containing molecule is an antibody or antibody fragment.

4. The method of claim 3, wherein said antibody or antibody fragment is a monoclonal antibody or monoclonal antibody fragment.

5. The method of claim 3, wherein said antibody or antibody fragment is a humanized antibody or humanized antibody fragment.

6. The method of claim 3, wherein said antibody or antibody fragment is a fully human antibody or a fully human antibody fragment.

7. The method of claim 3, wherein said antibody or antibody fragment binds specifically to a molecule found on surfaces of cancer cells.

8. The method of claim 6, wherein said molecule is A33.

9. The method of claim 5, wherein said humanized antibody or humanized antibody fragment binds specifically to a molecule found on surfaces of cancer cells.

10. The method of claim 8, wherein said molecule is A33.

11. The method of claim 1, wherein said sample is a serum, plasma, blood, urine, cerebrospinal fluid, sweat, tissue exudate, or tissue homogenate sample

12. In a method for determining efficacy of a therapeutic agent by determining presence of an immune agent specific for said therapeutic agent in a body fluid sample of a subject and comparing it to a value obtained from the same body fluid of said patient and determining differences there between as indicative of efficacy or lack thereof, the improvement comprising determining said presence via plasmon resonance.
